(19) United States
(12) Patent Application Publication
(10) Pub. No.: US 2020/0008894 A1
(43) Pub. Date: Jan. 9, 2020

(54) MEDICAL OPERATION SYSTEM, SURGICAL SYSTEM, SURGICAL INSTRUMENT, AND EXTERNAL FORCE SENSING SYSTEM

(71) Applicant: SONY CORPORATION, TOKYO (JP)

(72) Inventors: HIROYUKI SUZUKI, TOKYO (JP); KENICHIRO NAGASAKA, TOKYO (JP)

(21) Appl. No.: 16/490,189

(22) PCT Filed: Jan. 22, 2018
(86) PCT No.: PCT/JP2018/001841

§ 371 Date: Aug. 30, 2019

(30) Foreign Application Priority Data
Mar. 10, 2017 (JP) 2017-046789

(51) Int. Cl. A61B 34/37; A61B 10/06; A61B 90/00; G01L 5/16; B25J 3/00; B25J 13/08

(52) U.S. Cl. CPC A61B 34/37; A61B 10/06; A61B 90/06; G01L 5/166; A61B 2010/0208; B25J 13/085; A61B 2090/064; A61B 2090/065; B25J 3/00

(57) ABSTRACT

The present technology is to provide a medical operation system, a surgical system, a surgical instrument, and an external force sensing system that detect a force acting on an end effector in a preferred manner. The medical operation system includes: an inner slave having an end effector; an outer slave into which the inner slave is inserted, the outer slave supporting the inner slave at a position that allows the end effector to protrude outward from an end of the outer slave; a strain detection unit that detects strain generated in the outer slave; and a processing unit that calculates a force acting on the end effector in a living subject, on the basis of a result of detection performed by the strain detection unit. The outer slave is a structure decoupled from the inner slave.

### TECHNICAL FIELD

The technology disclosed in the present specification to provide a medical operation system, a surgical system, a surgical instrument, and an external force sensing system that detect a force acting on an end effector.

### BACKGROUND ART

Advances in the robotics technologies in recent years are remarkable, and robots are widely used in work sites in various industrial fields. For example, in a master-slave robot system, a person (an operator) handles a master arm in front of the person, and a slave arm located at a remote place traces the movement of the master arm. Thus, remote control of a manipulator can be realized. Master-slave robot systems such as medical robots are used in industrial fields where fully autonomous operations by computer control are still difficult.

For example, “da Vinci Surgical System (da Vinci)” manufactured by Intuitive Surgical Inc. of the United States is the first master-slave surgical robot that was developed for endoscopic surgery such as abdominal surgery and thoracic surgery. The surgical robot, “da Vinci”, is equipped with various kinds of robot forcipes, and further, the practitioner can perform surgery through remote control of the slave arm by recognizing the surgical field while watching a 3D monitor screen.

For this reason, several proposals have also been made for medical robotics systems capable of detecting a force acting on an end effector such as a gripping unit (gripper) (see Non-Patent Document 1, for example).

In a surgical robot to be used for endoscopic surgery, it is necessary to reduce the size of the configuration of the end effector, and a drive mechanism is normally used. By this drive mechanism, a driving force generated by a drive unit such as a motor disposed at a distance from the end effector is transmitted through a cable, to open and close the end effector. In the above described force-detectable medical robotics system, a force sensor is disposed between the end effector and the drive unit that drives the end effector. In such a configuration, the tractive force of the cable for opening and closing the end effector interferes with an external force applied in the long axis direction of the end effector, for example. This might lower the sensitivity of the force sensor, or make calibration difficult.

### CITATION LIST

### Non-Patent Document

Non-Patent Document 1: Ulrich Seibold et al., “Prototype of Instrument for Minimally Invasive Surgery with 6-Axis Force Sensing Capability”, Proceedings of the 2005 IEEE International Conference on Robotics and Automation, pp. 498-503, Barcelona, Spain, April 2005

### SUMMARY OF THE INVENTION

### Problems to be Solved by the Invention

An object of the technology disclosed in the present specification is to provide an excellent medical operation system, a surgical system, a surgical instrument, and an external force sensing system that are capable of detecting a force acting on an end effector in a preferred manner.

### Solutions to Problems

The technology disclosed in the present specification is made in view of the above problems, and a first aspect thereof is a medical operation system that includes:

an inner slave having an end effector;

an outer slave into which the inner slave is inserted, the outer slave supporting the inner slave at a position that allows the end effector to protrude outward from an end of the outer slave;

a strain detection unit that detects strain generated in the outer slave; and

a processing unit that calculates a force acting on the end effector in a living subject, on the basis of a result of detection performed by the strain detection unit.

It should be noted that the term “system” means a logical assembly of a plurality of devices (or functional modules that realize specific functions), and the respective devices or functional modules are not necessarily in a single housing.

Here, the outer slave has a bending portion that bends in a long axis direction, and the strain detection unit is disposed on a distal end side than the bending portion. Further, the outer slave has a structure decoupled from the inner slave, and a cable for pulling the end effector is inserted together with the inner slave into the outer slave.

The strain detection unit includes strain detection elements disposed at two positions on respective opposite sides in two directions perpendicular to the long axis direction of the outer slave. Specifically, the strain detection unit includes the strain detection elements including FBG sensors formed at the two positions on optical fibers attached to the respective opposite sides in the two directions perpendicular to the long axis direction of the outer slave. Further, dummy FBG sensors are formed in the optical fibers. Further, the outer slave has a shape that allows stress to concentrate at the two positions at which the strain detection elements are disposed.

The processing unit then calculates a translational force and a moment acting on the end effector, on the basis of strains at the two positions on the respective opposite sides in the two directions perpendicular to the long axis direction of the outer slave, the strains having been detected by the strain detection elements. The processing unit also calculates a translational force and a moment acting on the end effector, on the basis of strains at the two positions on the respective opposite sides in the two directions perpendicular to the long axis direction of the outer slave, the strains having been detected by the strain detection elements.

Further, the processing unit removes a strain component caused by a temperature change from the average value, and calculates a force acting in the long axis direction of the end effector. Specifically, the processing unit removes a strain component caused by a temperature change from a result of detection performed by the FBG sensors, on the basis of wavelength changes of the dummy FBG sensors.

Further, a second aspect of the technology disclosed in the present specification is a surgical system that includes:

a master device; and a slave device remotely controlled by the master device,

the slave device including

an inner slave having an end effector,

an outer slave into which the inner slave is inserted, the outer slave supporting the inner slave at a position that allows the end effector to protrude outward from an end of the outer slave,

a strain detection unit that detects strain generated in the outer slave,

a processing unit that calculates a force acting on the end effector in a living subject, on the basis of a result of detection performed by the strain detection unit, and

an output unit that outputs a result of processing performed by the processing unit, to the master device.

Further, a third aspect of the technology disclosed in the present specification is a surgical instrument that includes:

an inner slave having an end effector;

an outer slave into which the inner slave is inserted, the outer slave supporting the inner slave at a position that allows the end effector to protrude outward from an end of the outer slave;

a strain detection unit that detects strain generated in the outer slave; and

a transmission unit that transmits a result of detection performed by the strain detection unit.

Further, a fourth aspect of the technology disclosed in the present specification is an external force sensing system that includes:

an inner slave having an end effector;

an outer slave into which the inner slave is inserted, the outer slave supporting the inner slave at a position that allows the end effector to protrude outward from an end of the outer slave;

a strain detection unit that detects strain generated in the outer slave; and

a processing unit that calculates a force acting on the end effector, on the basis of a result of detection performed by the strain detection unit.

### Effects of the Invention

According to the technology disclosed in the present specification, it is possible to provide an excellent medical operation system, a surgical system, a surgical instrument, and an external force sensing system that are capable of detecting a force acting on an end effector in a preferred manner.

Note that the advantageous effects described in the present specification are merely examples, and the advantageous effects of the present invention are not limited to them. Furthermore, the present invention may exhibit additional advantageous effects, in addition to the above described advantageous effects.

Other objects, features, and advantages of the technology disclosed in the present specification will be made apparent by the embodiments described below and the detailed descriptions with reference to the accompanying drawings.

### BRIEF DESCRIPTION OF DRAWINGS

FIG. 1 is a diagram schematically showing an example configuration of a surgical system 100.

FIG. 2 is a diagram schematically showing an example configuration of the surgical system 100.

FIG. 3 is a diagram schematically showing an example configuration of the surgical system 100.

FIG. 4 is a diagram showing an example of forces acting on an end effector 111.

FIG. 5 is a diagram showing an example in which strain detection elements are attached to a first outer casing 121.

FIG. 6 is a diagram showing an example in which strain detection elements are attached to the first outer casing 121.

FIG. 7 is a diagram for explaining a mechanism for detecting forces acting on the first outer casing 121 (a cantilever).

FIG. 8 is a diagram for explaining a method of installing strain detection elements 2501a through 504a and 501b through 504b in the first outer casing 121, using FBG sensors.

FIG. 9 is a diagram showing an example configuration example of dummy FBG sensors.

FIG. 10 is a diagram showing a functional configuration for a signal processing unit 1000 to calculate translational forces and moments acting on the end effector 111.

FIG. 11 is a diagram schematically showing the functional configuration of a master-slave robot system 1100.

### MODE FOR CARRYING OUT THE INVENTION

The following is a detailed description of embodiments of the technology disclosed in the present specification, with reference to the drawings.

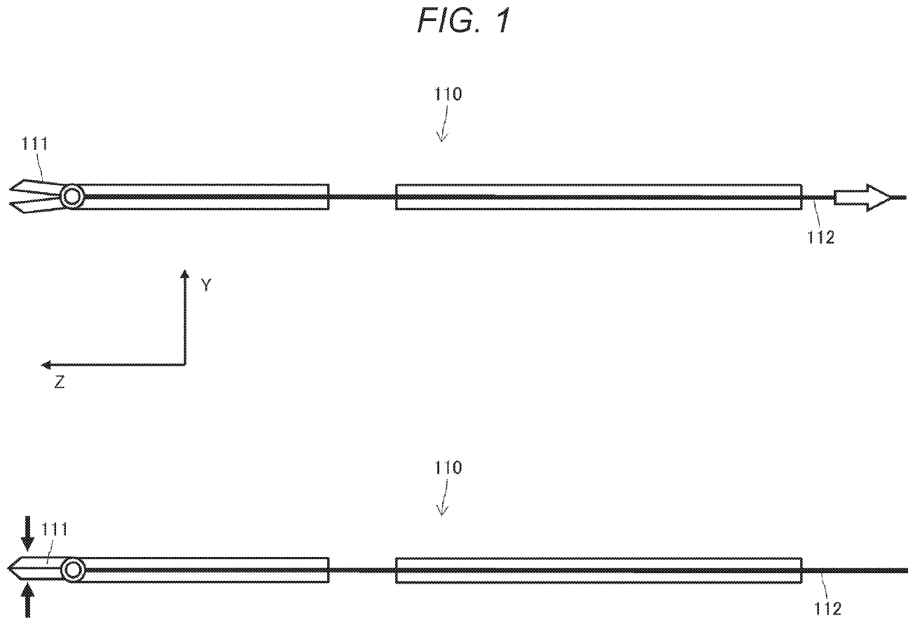

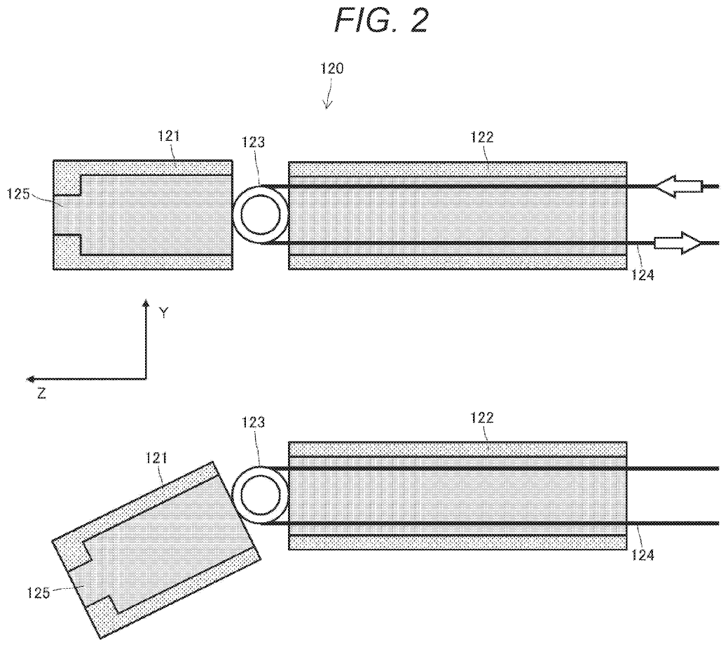

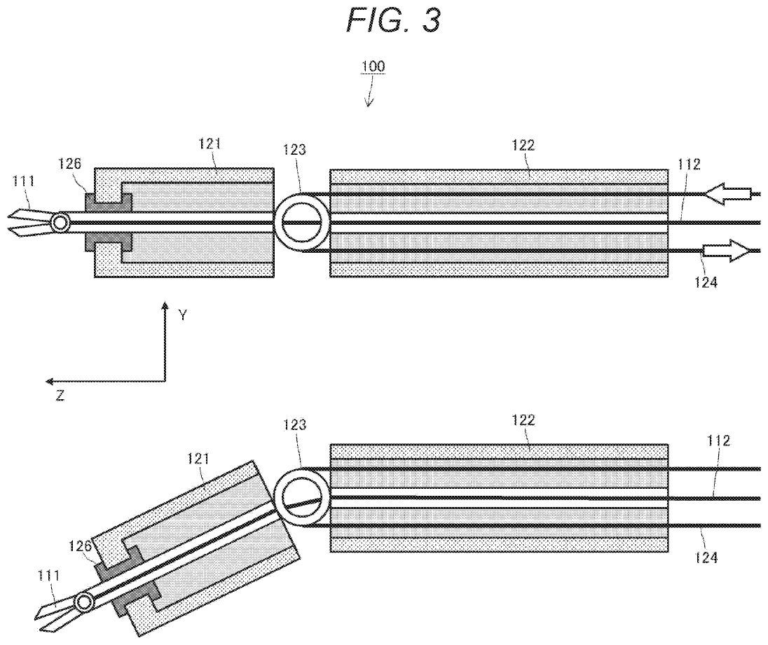

FIGS. 1 through 3 schematically show an example configuration example of a surgical system 100 to which the technology disclosed in the present specification can be applied. The surgical system 100 shown in the drawing includes a gripping mechanism unit 110 for gripping an object such as a body tissue or a surgical instrument, and an outer casing member 120 into which the gripping mechanism unit 110 is axially inserted. The surgical system 100 may also be regarded as a two-layer structure including the gripping mechanism unit 110 as an inner slave and the outer casing member 120 as an outer slave. FIG. 1 primarily shows the configuration of the gripping mechanism unit 110. FIG. 2 primarily shows the configuration of the outer casing member 120. FIG. 3 shows the entire configuration of the surgical system 100 in which the gripping mechanism unit 110 is inserted into the outer casing member 120.

In the description below, an X-Y-Z coordinate system is set, and the long axis direction of the gripping mechanism unit 110 is the Z-axis in the X-Y-Z coordinate system. Accordingly, the leftward direction in the drawing is the Z-axis, a direction perpendicular to the drawing is the X-axis, and a vertical direction in the drawing is the Y-axis.

FIG. 1 shows only the gripping mechanism unit 110. On the other hand, FIG. 2 shows a cross-section of only the outer casing member 120, taken along a plane parallel to the long axis direction (the Y-X plane). Further, FIG. 3 shows a cross section of the gripping mechanism unit 110 inserted into and secured in the outer casing member 120, taken along the plane parallel to the long axis direction (the Y-X plane).

The gripping mechanism unit 110 is equivalent to a treatment tool also called a “biopsy forceps”, and has an end effector 111 at its tip. The end effector 111 includes a pair of blades that can be opened and closed. The end effector 111 can be opened and closed by a tractive force transmitted from a drive unit (not shown) such as a motor via a cable 112, and grip an object such as a body tissue or a surgical instrument. In the example shown in FIG. 1, the end effector 111 can be closed by a tractive force of the cable 112, and grip an object.

Meanwhile, the outer casing member 120 is a guide tube equivalent to a “trocar”. The outer casing member 120 includes a hollow cylindrical structure, and is inserted into a body cavity such as an abdominal cavity or a chest cavity, to guide the gripping mechanism unit 110.

In a body cavity, it is not necessarily possible to move the gripping mechanism unit 110 (or the end effector 111) straight from the position at which the outer casing member 120 is inserted to the position at which the object to be gripped exists. For this reason, the outer casing member 120 has a bending structure so that it becomes possible to bypass an obstacle or the like and reach the object to be gripped from the position at which the outer casing member 120.

Specifically, as can be seen from FIG. 2, the outer casing member 120 is separated into a first outer casing 121 and a second outer casing 122 in this order from the distal end. Further, as the root of the first outer casing 121 is rotatably supported at the tip of the second outer casing 122 via a first joint 123, the outer casing member 120 can bend. When the first joint 123 rotates by virtue of a tractive force transmitted from a drive unit (not shown) such as a motor via a cable 124, the first outer casing 121 bends away from the long axis direction.

The surgical system 100 is equivalent to a biopsy forceps that is detachably mounted on a robot arm of a medical or surgical robot to be used for performing ophthalmic surgery, brain surgery, or endoscopic surgery such as abdominal surgery or thoracic surgery in a minimally invasive manner, for example. In a case where the surgical system 100 is a slave in a master-slave robot system, a drive unit for pulling the biopsy forceps, which is the end effector 111, with the cable 112, and a drive unit for pulling the first outer casing 121 with the cable 124 are activated in accordance with instructions from the master. Further, in the master-slave robot system, it is preferable to give a feedback of information about the position of the slave arm, the external force to be applied to the slave arm, and the like, so that the operator can perform remote control on the slave arm accurately and efficiently with the master arm, without damaging the target object.

Although not shown in the drawings, the surgical system 100 may also be designed so that the root of the second outer casing 122 is also rotatably supported by the tip of a third outer casing (not shown), and rotates by virtue of a tractive force of a cable.

The first outer casing 121 and the second outer casing 122 are guide tubes. Each of the guide tubes has a hollow cylindrical shape, and allows the gripping mechanism unit 110 to be inserted thereinto, to guide the gripping mechanism unit 110 in a body cavity like a “trocar”. An opening 125 for letting out the tip of the gripping mechanism unit 110 is formed almost at the center of the end face on the distal end side of the first outer casing 121. The gripping mechanism unit 110 is inserted into the hollow first outer casing 121 from the proximal end side. A portion with a predetermined length from the tip of the gripping mechanism unit 110 including the end effector 111 then protrudes from the opening 125 toward the outside. In such a positional relationship, the gripping mechanism unit 110 is supported by a support 126 so as to be rotatable about the long axis, at the opening 125 of the end edge of the first outer casing 121.

The surgical system 100 according to this embodiment can achieve one degree of freedom in gripping and one degree of freedom in bending, by combining the gripping mechanism unit 110 including the end effector 111 capable of opening and closing and the outer casing member 120 having a bending structure. Further, the gripping mechanism unit 110 as an inner slave has a degree of freedom in rotating about the long axis relative to the outer casing member 120 as an outer slave.

Note that the gripping mechanism unit 110 as an inner slave and the first outer casing 121 as an outer slave are decoupled from each other. Although shown in a simplified manner in FIG. 3, the support 126 includes a rolling bearing or a sliding bearing, for example, and rotatably supports the gripping mechanism unit 110 so as to be rotatable about the long axis relative to the outer casing 121. Therefore, the gripping mechanism unit 110 and the outer casing 121 are slidably independent of each other (or are floating from each other), with a predetermined fitting error being allowed. The gripping mechanism unit 110 can transmit the gripping force for the end effector 111 independently of the outer casing 121, and does not apply any external disturbance to the outer casing 121 when performing a gripping action. Further, the gripping mechanism unit 110 is assumed to have a flexible structure like a biopsy forceps, and has a degree of freedom in being deformed in the direction in which an external force acts. When deformed, the gripping mechanism unit 110 is brought into contact with the outer casing 121, and can transmit an external formed applied to the end effector 111, indirectly to the outer casing 121. In other words, because of the decoupled structure, a translational force acting on the end effector 111 at the tip of the gripping mechanism unit 110 also acts on the first outer casing 121, but the tractive force generated from the cable 112 for gripping the end effector 111 is not applied to the first outer casing 121.

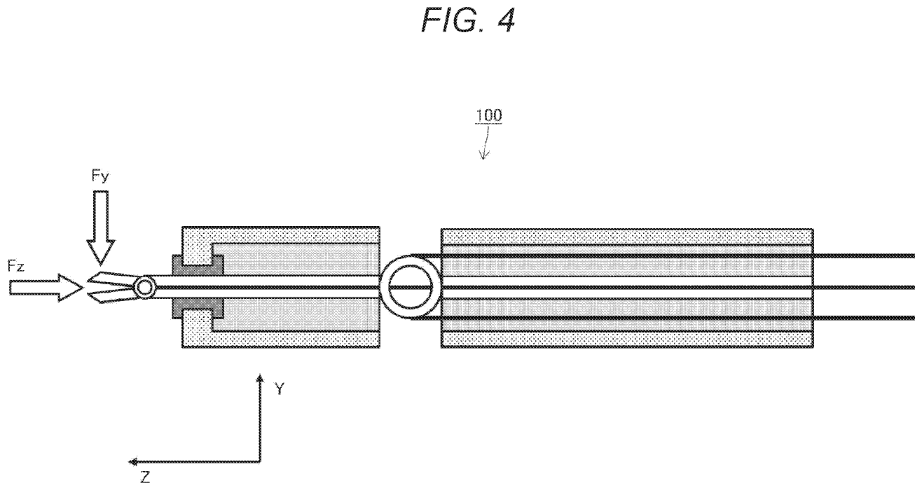

FIG. 4 shows an example of forces acting on the end effector 111. During an operation in a body cavity or the like, an external force Fz in the Z direction, external forces Fx and Fy in the X direction and the Y direction act on the end effector 111, and translational forces Fx and Fy in the X and Y directions and moments Mx and My about the X- and Y-axes also act on the end effector 111.

In a case where the surgical system 100 is applied to a slave device in a master-slave robot system, a force acting on the end effector 111 is detected, and may be used for force sense presentation to the operator on the master device side. Further, in a case where the end effector 111 opens and closes by virtue of a driving force transmitted via the cable 112, it is necessary to detect forces acting on the end effector 111, without interfering with the tractive force of the cable 112.

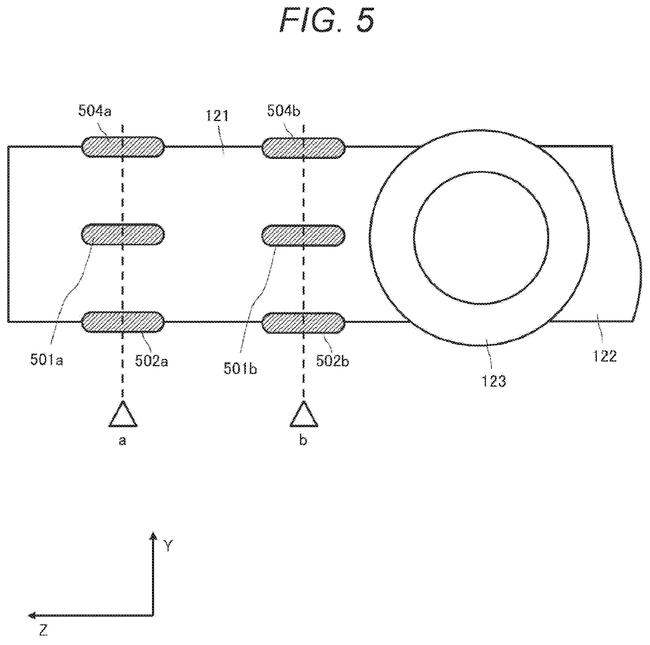

FIG. 5 schematically shows a configuration for detecting forces acting on the end effector 111 in the surgical system 100 shown in FIGS. 1 through 3.

The gripping mechanism unit 110 is supported by the support 126 so as to be rotatable about the long axis direction relative to the first outer casing 121 (as described above). The translational forces acting on the end effector 111 also act on the first outer casing 121. As a result, the first outer casing 121 generates a strain Δεin accordance with the translational forces Fx, Fy, and Fz acting on the end effector 111.

The first outer casing 121 may be regarded as a cantilever that bends in the X and Y directions, and expands and contracts in the Z direction, with the first joint 123 being the fixed end. Therefore, in this embodiment, the first outer casing 121 is used as a strain generator, and a strain detection element is disposed at one or more locations on the outer periphery of the first outer casing 121. In the example shown in FIG. 5, a plurality of strain detection elements for detecting strains in the X and Y directions at two different positions a and b in the long axis direction is attached to the outer periphery of the first outer casing 121.

Specifically, at the position a, a pair of strain detection elements 501a and 503a (not shown) for detecting an amount of strain of the first outer casing 121 in the X direction is attached to opposite sides of the outer periphery of the first outer casing 121. Further, a pair of strain detection elements 502a and 504a for detecting an amount of strain of the first outer casing 121 in the Y direction is attached to opposite sides of the outer periphery of the first outer casing 121. Likewise, at the position b, a pair of strain detection elements 501b and 503b (not shown) for detecting an amount of strain of the first outer casing 121 in the X direction is attached, and a pair of strain detection elements 502b and 504b for detecting an amount of strain in the Y direction is attached.

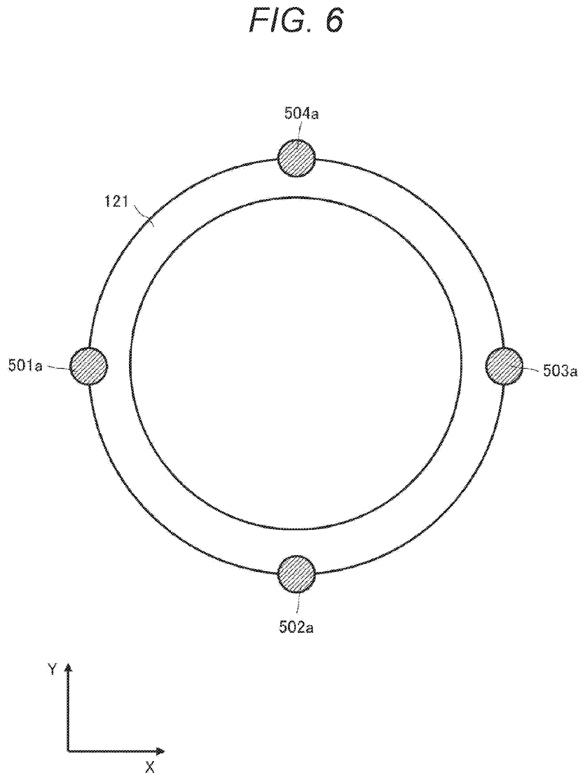

FIG. 6 shows an X-Y cross-section of the first outer casing 121 at the position a. As can be seen from this drawing, the pair of strain detection elements 501a and 503a for detecting an amount of strain in the X direction is attached to opposite sides in the X direction of the outer periphery of the first outer casing 121, and the pair of strain detection elements 502a and 504a for detecting an amount of strain in the Y direction is attached to opposite sides in the Y direction of the outer periphery of the first outer casing 121. Note that, although not shown in the drawing, in a X-Y cross-section of the first outer casing 121 at the position b, the pair of strain detection elements 501b and 503b for detecting an amount of strain in the X direction is attached to opposite sides in the X direction of the outer periphery of the first outer casing 121, and the pair of strain detection elements 502b and 504b for detecting an amount of strain in the Y direction is attached to opposite sides in the Y direction of the outer periphery of the first outer casing 121, as in FIG. 6.

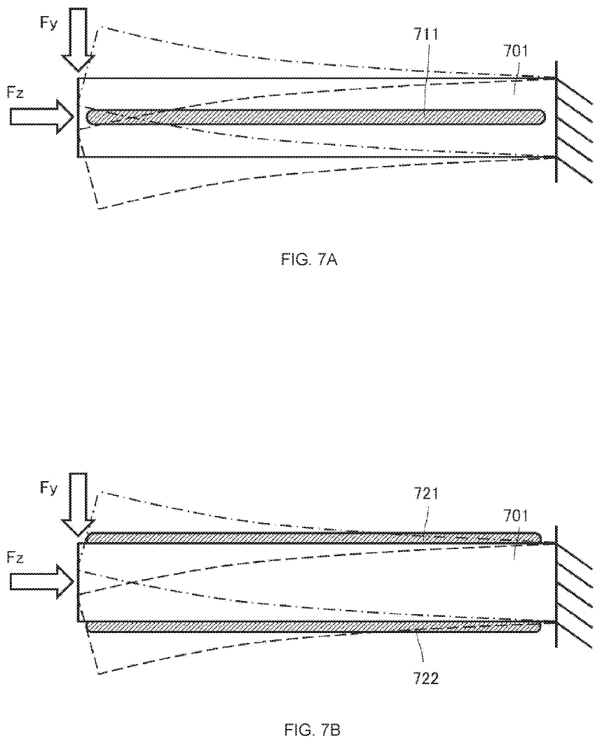

First, the reason that the pair of strain detection elements 501a and 503a (or 501b and 503b) are disposed on opposite sides in the X direction, and the pair of strain detection elements 502a and 504a (or 502b and 504b) are disposed on opposite sides in the Y direction at one detection position is described below, with reference to FIG. 7.

As shown in FIG. 7(A), in a case where only one strain detection element 711 is attached to a cantilever 301, when an external force Fz in the Z direction is applied to the cantilever 701, the strain detection element 711 contracts, and accordingly, the external force Fz can be measured. However, the strain detection element 711 expands regardless of whether the cantilever 701 bends upward or downward on the paper surface. Therefore, it is not possible to determine, only from a result of detection performed by the strain detection element 711, whether the direction in which an external force Fy applied in the Y direction acts is positive or negative (upward or downward on the paper surface).

On the other hand, in a case where a pair of detection elements 721 and 722 are attached to opposite sides of the cantilever 701 in the Y direction, as shown in FIG. 7(B), when the cantilever 701 bends upward on the paper surface, the strain detection element 721 contracts, and the other strain detection element 722 expands. Conversely, when the cantilever 701 bends downward on the paper sheet, the strain detection element 721 expands, and the other strain detection element 722 contracts. Accordingly, it is possible to detect the direction in which the external force Fy applied in the Y direction acts, from the relationship between the positive and negative signs of the strain amounts detected by the pair of detection elements 721 and 722 attached to opposite sides in the Y direction.

Because of this, the sum of the strain amounts detected by the pair of strain detection elements 501a and 503a (or 501b and 503b) attached to opposite sides in the X direction at a position in the long axis direction of the first outer casing 121 is calculated, so that the external force in the Z direction acting on the first outer casing 121 can be detected, and it also becomes possible to calculate the external force in the X direction acting on the first outer casing 121 by obtaining the difference between the respective strain amounts. Further, the strain amounts detected by the respective strain detection elements 501a and 503a (or 501b and 503b) each include not only a component derived from the acting force but also a component derived from temperature change. However, in a case where the external force in the X direction is calculated from the difference between the respective strain amounts, the component derived from temperature change is cancelled, and there is no need to perform a temperature compensation process. Note that the method of performing temperature compensation by calculating the detection value difference between sensors installed on opposite sides is also known as a four-gauge method using four strain gauges, for example, in this field of technology.

Likewise, the sum of the strain amounts detected by the pair of strain detection elements 502a and 504a (or 502b and 504b) attached to opposite sides in the Y direction at a position in the long axis direction of the first outer casing 121 is calculated, so that the external force in the Z direction acting on the first outer casing 121 can be detected, and it also becomes possible to calculate the external force in the Y direction acting on the first outer casing 121 by obtaining the difference between the respective strain amounts. Further, the strain amounts detected by the respective strain detection elements 502a and 504a (or 502b and 504b) each include not only a component derived from the acting force but also a component derived from temperature change. However, in a case where the external force in the Y direction is calculated from the difference between the respective strain amounts, the component derived from temperature change is cancelled, and there is no need to perform a temperature compensation process (same as above).

Next, the reason that the configuration in which the amounts of strain in the X and Y directions are detected at the two positions a and b different in the long axis direction of the first outer casing 121 is described.

The translational force can be calculated from the amount of strain at one point on a cantilever, but the moment is not calculated from the amount of strain. On the other hand, the moment as well as the translational force can be calculated from the amounts of strain at two or more positions. Accordingly, with the configuration shown in FIG. 5, the translational force Fx in the X direction acting on the first outer casing 121 and the moment Mx about the X-axis can be calculated on the basis of the amounts of strain in the X direction detected at the two positions a and b. Likewise, the translational force Fy in the Y direction acting on the first outer casing 121 and the moment My about the Y-axis can be calculated on the basis of the amounts of strain in the Y direction detected at the two positions a and b.

The entire surgical system 100 can be regarded as being equipped with a sensor having 5 degrees of freedom (DOF) including the moments Mx and My about the two axes, in addition to the translational forces Fx, Fy, and Fz in the three directions.

A tractive force of the cable 112 for opening and closing the end effector 111 acts on the gripping mechanism unit 110 inserted into the first outer casing 121. However, since the gripping mechanism unit 110 as the inner slave and the first outer casing 121 as the outer slave are decoupled from each other (described above), the tractive force of the cable 112 does not act on the first outer casing 121. Accordingly, the 5-DOF sensor mounted on the first outer casing 121 does not interfere with the tractive force of the cable 112 (in other words, the gripping force of the end effector 111), and thus, the acting forces Fx, Fy, and Fz of the 5-DOF acting on the end effector 110, and the moments Mx and My can be measured with high sensitivity. In addition to the above, there is an effect to reduce mechanical vibration noise, as the actual inertia in the stage after the 5-DOF sensor is reduced.

In FIG. 2 through FIG. 4, FIG. 6, and others, the first outer casing 121 is shown as a simple cylindrical structure, for simplification of the drawings. As the first outer casing 121 has a suitable structure as a strain generator, detection performance of the 5-DOF sensor is improved. In other words, as the first outer casing 121 is formed into such a shape that stress concentrates on each of the two measurement positions a and b in the long axis direction, and deformation is easily caused, the amounts of strain can be easily measured by the strain detection elements 501a through 504a and 501b through 504b, and detection performance of the 5-DOF sensor is expected to become higher.

Meanwhile, strain detection elements widely known in the industry include capacitive sensors, semiconductor strain gauges, and foil strain gauges, any of which can be used as the strain detection elements 501a through 504a and 501b through 504b. In this embodiment, however, fiber bragg grating (FBG) sensors manufactured using optical fibers are used as the strain detection elements 501a through 504a and 501b through 504b.

Here, an FBG sensor is a sensor formed by cutting a diffraction grating (a grating) along the long axis of an optical fiber, and is capable of detecting a change in the intervals between diffraction gratings due to expansion or contraction accompanying strain or temperature change caused by an acting force, and regarding the change in the intervals as a change in the wavelength of reflected light of incident light of a predetermined wavelength band (Bragg wavelength). The change in the wavelength detected from the FBG sensor can be then converted into strain, stress, or temperature change, which is the cause.

In this embodiment, it is assumed that a signal processing unit that processes a detection signal is disposed at a location at a distance from the first outer casing 121 to which the strain detection elements 501a through 504a and 501b through 504b are attached. An FBG sensor using an optical fiber has small transmission loss (or is not easily affected by noise from the outside), and thus, can maintain high detection accuracy under any conceivable environment. Further, an FBG sensor also has the advantage of being capable of coping with sterilization and high magnetic field environments that are necessary for medical treatment.

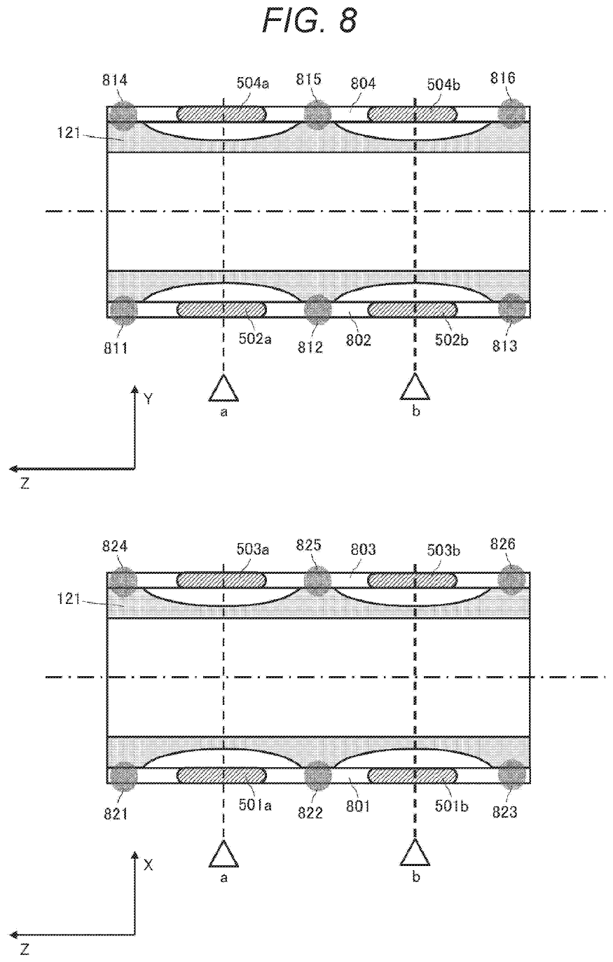

The structure of the first outer casing 121 designed to be easily deformed at the two measurement positions a and b, and a method of disposing the strain detection elements 501a through 504a and 501b through 504b using FBG sensors on the outer periphery of the first outer casing 121 are now described, with reference to FIG. 8.

FIG. 8 shows a Y-Z cross-section and a Z-X cross-section of the first outer casing 121. In this drawing, the portions of the Y-Z cross-section and the Z-X cross-section of the first outer casing 121 are shaded. It is to be understood that the first outer casing 121 is hollow and rotationally symmetrical about its long axis. Note that, although the gripping mechanism unit 110 is inserted into the inside of the hollow, the gripping mechanism unit 110 is not shown in FIG. 8, for simplification.

As shown in the drawing, the outer periphery of the first outer casing 121 has a constricted structure that has concave portions at which the radius becomes gradually smaller at the two measurement positions a and b different in the long axis direction. On the other hand, the inner diameter of the first outer casing 121 is constant in the long axis direction, and the thickness of the concave portions is smaller. Accordingly, when a force is applied in at least one of the X or Y direction, the first outer casing 121 is easily deformed with stress concentrated at each of the measurement positions a and b, and can be used as a strain generator.

The first outer casing 121 is formed with stainless steel (steel use stainless: SUS), a Co-Cr alloy, or a titanium-based material known as a metal-based material that excels in biocompatibility, for example, To form a strain generator in a portion of the structure as described above, it is preferable to manufacture the first outer casing 121, using a material having mechanical characteristics such as high strength and low rigidity (a low Young's modulus), like a titanium alloy, for example. Using a low-rigidity material as the strain generator, it is possible to measure forces acting on the end effector 111 with high sensitivity. Meanwhile, a titanium alloy has biocompatibility, and is a preferred material for use in medical settings such as surgery.

On the outer periphery of the first outer casing 121, a pair of optical fibers 802 and 804 are laid in the long axis direction on opposite sides in the Y direction. Likewise, on the outer periphery of the first outer casing 121, a pair of optical fibers 801 and 803 are laid in the long axis direction on opposite sides in the X direction. In short, four optical fibers 801 through 804 are laid in the entire first outer casing 121.

Of the optical fibers 802 and 804 laid on opposite sides in the Y direction, the portions overlapping with the two concave portions of the first outer casing 121 (or near the measurement positions a and b) are cut away from the diffraction grating, and FBG sensors are formed. The respective FBG sensors are then used as the strain detection elements 502a, 502b, 504a, and 504b. The portions of the optical fibers 802 and 804 in which the FBG sensors are formed are shaded in the drawing.

Further, the respective optical fibers 802 and 804 are fixed to the surface of the first outer casing 121 with an adhesive or the like at both ends 811 through 813 and 814 through 816 of the portions in which the FBG sensors 502a, 502b, 504a, and 504b are formed. Therefore, when an external force acts on the first outer casing 121, and the first outer casing 121 bends in the Y direction, the respective optical fibers 802 and 804 are also integrally deformed, and strains are generated in the FBG sensor portions, which are the strain detection elements 502a, 502b, 504a, and 504b.

Likewise, of the optical fibers 801 and 803 laid on opposite sides in the X direction, the portions overlapping with the two concave portions of the first outer casing 121 (or near the measurement positions a and b) are cut away from the diffraction grating, and FBG sensors are formed. The respective FBG sensors are then used as the strain detection elements 501a, 501b, 503a, and 503b. The portions of the optical fibers 801 and 803 in which the FBG sensors are formed are shaded in the drawing.

Further, the respective optical fibers 801 and 801 are fixed to the surface of the first outer casing 121 with an adhesive or the like at both ends 821 through 823 and 824 through 826 of the portions in which the FBG sensors 501a, 501b, 503a, and 503b are formed. Therefore, when an external force acts on the first outer casing 121, and the first outer casing 121 bends in the Y direction, the respective optical fibers 801 and 803 are also integrally deformed, and strains are generated in the FBG sensor portions, which are the strain detection elements 501a, 501b, 503a, and 503b.

Of the optical fibers 801 through 804 used as the strain detection elements 501a through 504a and 501b through 504b, only the portions attached to the outer periphery of the first outer casing 121 are shown in FIG. 8, and the other portions are not shown.

For example, dummy FBG sensors may be formed in portions separated from the outer periphery of the first outer casing 121, of the optical fibers 801 through 804 used as the strain detection elements 501a through 504a and 501b through 504b.

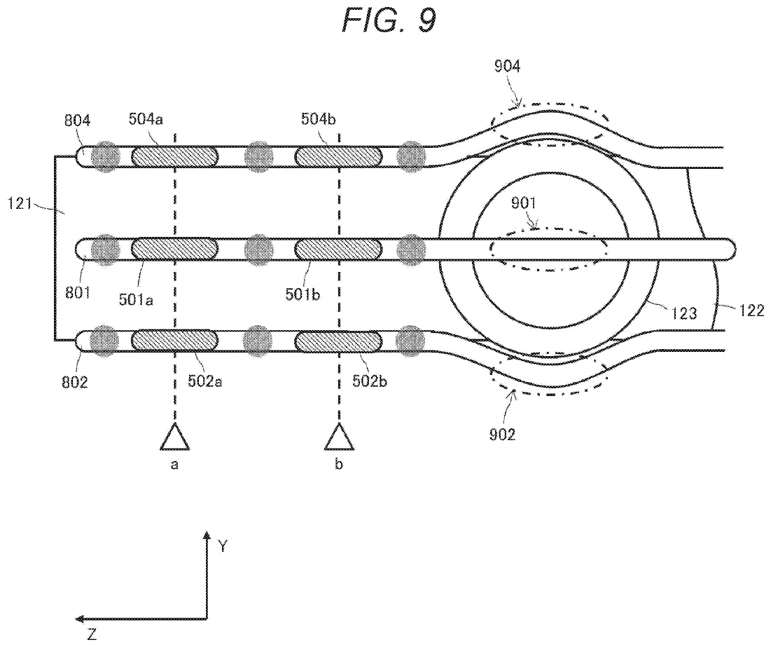

FIG. 9 shows an example in which dummy FBG sensors are disposed in the optical fibers 801, 802, and 804 attached to the outer periphery of the first outer casing 121. In the example shown in the drawing, as indicated by reference numerals 901, 902, and 904, the portions at which the respective optical fibers 801, 802, and 804 straddle the first joint 123 are cut away from the diffraction grating, and dummy FBG sensors are formed in the respective portions. Note that, although the optical fiber 503 is hidden and is not shown in the drawing, a dummy FBG sensor is also similarly disposed in the portion straddling the first joint 123.

As can be seen from FIG. 9, the dummy FBG sensors 901, 902, and 904 are formed in portions of the optical fibers 801, 802, and 804 not fixed to the outer periphery of the first outer casing 121 (in other words, the portions not fixed to the strain generator). Accordingly, it is possible to presume that wavelength changes detected by the respective dummy FBG sensors 901, 902, and 904 are wavelength changes that are not affected by strain of the first outer casing 121 and are affected only by changes in temperature.

Since the strain detection elements 501a through 504a and 501b through 504b are disposed on opposite sides in the X and Y directions, the component derived from a change in temperature is cancelled by the difference between the amounts of strain on opposite sides at the time of calculation of the translational forces Fx and Fy in the X and Y directions. Because of this, there is no need to perform a temperature compensation process (described above). On the other hand, when the translational force Fz in the Z direction is calculated, a temperature compensation process is only required to be performed with the use of wavelength changes Δλₜₑₘₚ of the dummy FBG sensors 901, 902, and 904.

Although only the portions of the optical fibers 801 through 804 attached to the outer periphery of the first outer casing 121 are shown in FIG. 8, the other ends extend to the detection unit and the signal processing unit (both are not shown in the drawing) beyond the first joint 123. The total length of the optical fibers 801 through 804 is assumed to be about 400 mm in practice, for example.

The detection unit and the signal processing unit are disposed at a location separated from the end effector 111, such as a position in the vicinity of the root of the surgical system 100, for example. The detection unit causes light of a predetermined wavelength (Bragg wavelength) to enter the optical fibers 801 through 804, and receives the reflected light to detect a change Δλ in wavelength. The signal processing unit then calculates the translational forces Fx, Fy, and Fz in three directions acting on the end effector 111 and moments Mx and My in two directions, on the basis of wavelength changes detected by the respective FBG sensors serving as the strain detection elements 501a through 504a and 501b through 504b that are attached to opposite sides of the first outer casing 121 in the X and Y directions and face one another. This arithmetic process to be performed by the signal processing unit will be described later in detail.

Most of the explanation made so far concerns the structure of the surgical system 100 according to this embodiment. Next, a processing algorithm to be executed by the signal processing unit is described. This processing algorithm is designed for calculating forces acting on the end effector 111 inserted into the first outer casing 121, on the basis of detection signals from the 5-DOF sensor formed on the first outer casing 121.

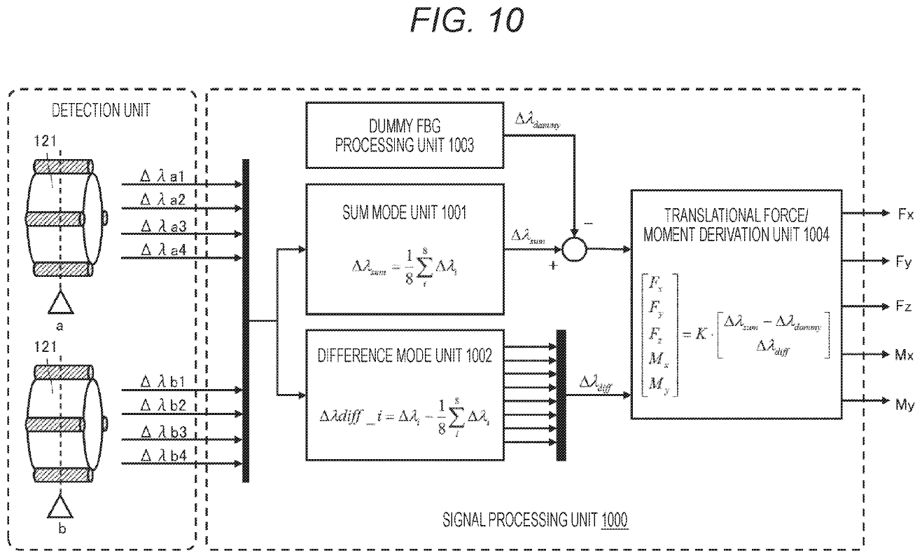

FIG. 10 schematically shows a 5-DOF sensor processing algorithm to be executed by a signal processing unit 1000 to calculate the translational forces Fx, Fy, and Fz in the three directions and the moments Mx and My acting on the end effector 111, on the basis of detection results obtained from the FBG sensors formed in the respective optical fibers 801 through 804 laid in the first outer casing 121.

On the basis of reflected light of incident light of a predetermined wavelength that enters the optical fibers 801 through 804 attached to the respective opposite sides of the first outer casing 121 in the X and Y directions, the detection unit detects wavelength changes Δλa1 through Δλa4 in the respective FBG sensors serving as the strain detection elements 501a through 504a laid at the position a on the first outer casing 121 when an external force acts on the end effector 111. However, the wavelength changes Δλa1 through Δλa4 detected also include wavelength change components derived from temperature changes.

On the basis of reflected light of incident light of a predetermined wavelength that enters the optical fibers 801 through 804 attached to the respective opposite sides of the first outer casing 121 in the X and Y directions, the detection unit also detects wavelength changes Δλb1 through Δλb4 in the respective FBG sensors serving as the strain detection elements 501b through 504b laid at the position a on the first outer casing 121 when an external force acts on the end effector 111. However, the wavelength changes Δλb1 through Δλb4 detected also include wavelength change components derived from temperature changes.

Although not shown in FIG. 10, the detection unit further detects wavelength changes in the dummy FBG sensors (see FIG. 9) formed in the respective optical fibers 801 through 804. The signal processing unit 1000 in the latter stage is designed to use the sum of the detection values obtained from the dummy FBG sensors or the value obtained by multiplying the total value by a calibration gain, as a wavelength change amount Δλ_{dammy} of the dummy FBG sensors (described later). The wavelength change amount Δλ_{dammy} is a wavelength change component derived from temperature changes in the respective optical fibers 801 through 804.

Here, the wavelength changes Δλa1 through Δλa4 detected by the detection unit from the positions a of the respective optical fibers 801 through 804 are equivalent to strain amounts Δεa1 through Δεa4 generated at the position a on the first outer casing 121 when an external force acts on the end effector 111. Meanwhile, the wavelength changes Δλb1 through Δλb4 detected by the detection unit from the positions b of the respective optical fibers 801 through 804 are equivalent to strain amounts Δεb1 through Δεb4 generated at the position b on the first outer casing 121 when an external force acts on the end effector 111 (in a case where the wavelength change components derived from temperature changes are ignored).

When the translational force Fx in the X direction or the moment Mx is generated in the end effector 111, the strain directions become opposite between the strain detection elements 501a and 503a and between the strain detection elements 501b and 503b disposed on opposite sides in the X direction, as can be seen from FIG. 7 (in other words, in a case where one element contracts, the other element expands). Further, with respect to the translational force Fx in the X direction or the moment Mx acting on the end effector 111, the strain directions are the same between the strain detection elements 502a and 504a and between the strain detection elements 502b and 504b disposed on opposite sides in the Y direction.

Likewise, when the translational force Fy in the Y direction or the moment My is generated in the end effector 111, the strain directions become opposite between the strain detection elements 502a and 504a and between the strain detection elements 502b and 504b disposed on opposite sides in the Y direction (in other words, in a case where one element contracts, the other element expands). Further, with respect to the translational force Fy in the Y direction or the moment My acting on the end effector 111, the strain directions are the same between the strain detection elements 501a and 503a and between the strain detection elements 501b and 503b disposed on opposite sides in the X direction.

Accordingly, as the differences among the wavelength changes Δλa1 through Δλa4 and Δλb1 through Δλb4 detected from the FBG sensors on opposite sides at the positions a and b on the respective optical fibers 801 through 804 are obtained, the wavelength change components derived from the translational forces Fx and Fy in the X and Y directions and the moments Mx and My acting on the end effector 111 can be extracted.

On the other hand, when the translational force Fz in the Z direction is generated in the end effector 111, the strain directions are the same in all the strain detection elements 501a through 504a and 501b through 504b. Accordingly, as the sum of the wavelength changes Δλa1 through Δλa4 and Δλb1 through Δλb4 detected from the positions a and b on the respective optical fibers 801 through 804 is obtained, the wavelength change components derived from the translational force Fz in the Z direction acting on the end effector 111 can be extracted.

A sum mode unit 1001 in the signal processing unit 1000 calculates the sum of the wavelength changes Δλᵢ detected from the positions a and b on the respective optical fibers 801 through 804 as shown in the following equation (1), and outputs the value obtained by dividing the sum by the number of strain detection elements (or the number of FBG sensors), which is eight.

$$\begin{matrix}[ Mathematical Formula 1 ] & \\ Δ {λ}_{sum} = \frac{1}{8} {\sum }_{i}^{8} Δ {λ}_{i} & ( 1 )\end{matrix}$$

However, the sum of the wavelength changes of the respective strain detection elements 501a through 504a and 501b through 504b contains wavelength change components derived from temperature changes, as well as the components derived from strains generated by the acting forces. Therefore, a dummy FBG processing unit 1003 obtains the sum of the detection values of the four dummy FBG sensors formed in the respective optical fibers 801 through 804 or the value obtained by multiplying the value of the sum by the calibration gain, and outputs the obtained value as the wavelength change amount Δλ_{dammy} detected by the dummy FBG sensors. The output Δλ_{dammy} of the dummy FBG processing unit 1003 is then subtracted from the output of the sum mode unit 1001, and thus, temperature compensation is performed.

Meanwhile, a difference mode unit 1002 subtracts the average value of these eight inputs from each of the eight inputs Δλa1 through Δλa4 and Δλb1 through Δλb4 obtained from the detection unit according to the following equation (2), and the subtraction result is output to a translational force-moment derivation unit 1004 in the later stage. The wavelength changes detected at the respective positions a and b include the wavelength change components Δλₜₑₘₚ derived from temperature changes, as well as the wavelength change components derived from acting strains generated by the translational forces Fx and Fy and the moments Mx and My. As the differential mode unit 1301 calculates the differences among the wavelength changes detected by the FBG sensors on opposite sides, it is possible to cancel the wavelength change components Δλₜₑₘₚ derived from temperature changes.

$$\begin{matrix}[ Mathematical Formula 2 ] & \\ Δ λ diff\_i = Δ {λ}_{i} - \frac{1}{8} {\sum }_{i}^{8} Δ {λ}_{{i}_{t}} & ( 2 )\end{matrix}$$

The translational force/moment derivation unit 1004 then multiplies the result of the temperature compensation process performed on the output of the sum mode unit 1001 (Δλₛᵤₘ−Δο_{dammy}) and the vector formed with the output Δλ_{diff} of the difference mode unit 1002 by a calibration matrix K, to calculate the translational forces Fx, Fy, and Fz, and the moments Mx and My acting on the end effector 111, as shown in the following equation (3).

$$\begin{matrix}[ Mathematical Formula 3 ] & \\ [ \begin{matrix}{F}_{x} \\ {F}_{y} \\ {F}_{z} \\ {M}_{x} \\ {M}_{y}\end{matrix} ] = K \cdot [ \begin{matrix}Δ {λ}_{sum} - Δ {λ}_{dammy} \\ Δ {λ}_{diff}\end{matrix} ] & ( 3 )\end{matrix}$$

Note that the calibration matrix K to be used in the calculation by the signal processing unit 1000 shown in FIG. 10 can be obtained through a calibration experiment, for example.

As described above, according to this embodiment, the surgical system 100 can detect the translational forces Fx, Fy, and Fz and the moments Mx and My acting on the end effector 111, with the 5-DOF sensor formed in the outer casing member 120 into which the gripping mechanism unit 110 having the end effector 111 is inserted. Further, as the gripping mechanism unit 110 and the outer casing member 120 are decoupled from each other (described above), it is possible to detect forces acting on the end effector 111, without interfering with the tractive force of the cable 112 for opening and closing the end effector 111.

For example, in a case where the surgical system 100 operates as a slave device in a master-slave robot system, detection results from the 5-DOF sensor described above are transmitted as feedback information about remote control to the master device. On the master device side, the feedback information can be used for various purposes. For example, the master device can perform force sense presentation to the operator, on the basis of the feedback information from the slave device. In the case of surgery, for example, it is possible to prevent organ damage by detecting an external force acting on the surgical system 100 and feeding it back to the operator (the surgeon) who uses the master device.

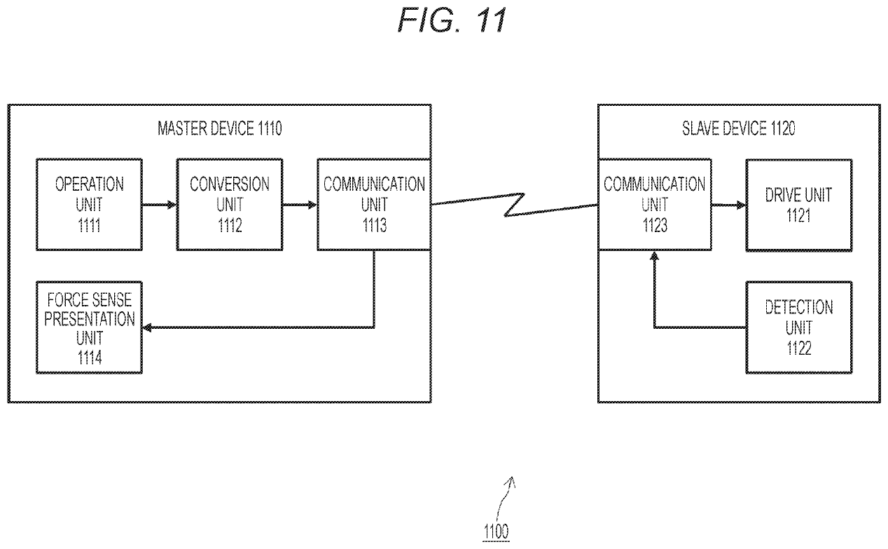

FIG. 11 schematically shows the functional configuration of a master-slave robot system 1100. The robot system 1100 includes a master device 1110 being operated by the operator, and a slave device 1120 being remotely controlled from the master device 1110 in accordance with an operation by the operator. The master device 1110 and the slave device 1120 are interconnected via a wireless or wired network.

The master device 1110 includes an operation unit 1111, a conversion unit 1112, a communication unit 1113, and a force sense presentation unit 1114.

The operation unit 1111 includes a master arm or the like for the operator to remotely control the slave device 1120. The conversion unit 1112 converts the contents of an operation performed by the operator on the operation unit 1111 into control information for controlling the driving on the side of the slave device 1120 (or more specifically, a drive unit 1121 in the slave device 1120).

The communication unit 1113 is mutually connected to the side of the slave device 1120 (or more specifically, a communication unit 1123 in the slave device 1120) via a wireless or wired network. The communication unit 1113 transmits the control information output from the conversion unit 1112, to the slave device 1120.

Meanwhile, the slave device 1120 includes the drive unit 1121, a detection unit 1122, and the communication unit 1123.

The slave device 1120 is assumed to be an arm-like robot that has a multi-link configuration and has the end effector 111 such as a multiaxial forceps attached to its tip as shown in FIG. 1. The drive unit 1121 includes a motor for rotationally driving the respective joints connecting links, and a motor for opening and closing the end effector 111. The motor for opening and closing the end effector 111 is disposed at a distance from the end effector 111, and a driving force is transmitted through the cable 112.

The detection unit 1122 is a 5-DOF sensor that is formed in the first outer casing 121, and is capable of detecting the translational forces Fx, Fy, and Fx in three direction and the moments Mx and My about the X- and Y-axes acting on the end effector 111.

The communication unit 1123 is mutually connected to the side of the master device 1110 (more specifically, the communication unit 1113 in the master device 1120) via a wireless or wired network. The drive unit 1121 mentioned above performs driving in accordance with the control information received by the communication unit 1123 from the side of the master device 1110. Further, the detection results (Fx, Fy, Fz, Mx, and My) obtained by the detection unit 1122 are transmitted from the communication unit 1123 to the side of the master device 1110.

On the side of the master device 1110, the force sense presentation unit 1114 performs force sense presentation to the operator, on the basis of the detection results (Fx, Fy, Fz, Mx, and My) received as feedback information by the communication unit 1113 from the slave device 1120.

Through the force sense presentation unit 1114, the operator operating the master device 1110 can recognize a contact force applied to the end effector on the side of the slave device 1120. For example, in a case where the slave device 1120 is a surgical robot, the operator appropriately performs adjustment during an operation with sutures by obtaining a tactile sensation such as a responding action on the forceps unit 110. Thus, closure can be completed, and efficient procedures can be conducted while invasion to the living tissue is prevented.

### INDUSTRIAL APPLICABILITY

The technology disclosed in the present specification has been described in detail, with reference to specific embodiments. However, it is obvious that those skilled in the art can make modifications to and substitutions of the embodiments without departing from the scope of the technology disclosed in the present specification.

The technology disclosed in the present specification can also similarly be applied to robotic devices of various types other than the master-slave type. Further, in the present specification, an embodiment in which the technology disclosed in the present specification is applied to a surgical robot has been primarily described. However, the scope of the technology disclosed in the present specification is not limited to this embodiment, and may also similarly be applied to robot devices that are to be used for medical purposes other than surgery, or in various fields other than the field of medicine.

In short, the technology disclosed in the present specification has been described through examples, and the descriptions in this specification should not be interpreted in a restrictive manner. The claims should be taken into account in understanding the subject matter of the technology disclosed in the present specification.

Note that the technology disclosed in the present specification may also be embodied in the configurations described below.

(1) A medical operation system including:

an inner slave having an end effector;

an outer slave into which the inner slave is inserted, the outer slave supporting the inner slave at a position that allows the end effector to protrude outward from an end of the outer slave;

a strain detection unit that detects strain generated in the outer slave; and

a processing unit that calculates a force acting on the end effector in a living subject, on the basis of a result of detection performed by the strain detection unit.

(2) The medical operation system according to (1), in which

the outer slave has a bending portion that bends in a long axis direction, and

the strain detection unit is disposed on a distal end side than the bending portion.

(3) The medical operation system according to (1) or (2), in which

the outer slave has a structure decoupled from the inner slave, and a cable for pulling the end effector is inserted together with the inner slave into the outer slave.

(4) The medical operation system according to any one of (1) to (3), in which

the strain detection unit includes strain detection elements disposed at two positions on respective opposite sides in two directions perpendicular to the long axis direction of the outer slave, and

the processing unit calculates a translational force and a moment acting on the end effector, on the basis of strains at the two positions on the respective opposite sides in the two directions perpendicular to the long axis direction of the outer slave, the strains having been detected by the strain detection elements.

(5) The medical operation system according to (4), in which

the strain detection unit includes the strain detection elements including FBG sensors formed at the two positions on optical fibers attached to the respective opposite sides in the two directions perpendicular to the long axis direction of the outer slave.

(6) The medical operation system according to (5), in which

dummy FBG sensors are formed in the optical fibers, and

the processing unit removes a strain component caused by a temperature change from a result of detection performed by the FBG sensors, on the basis of wavelength changes of the dummy FBG sensors.

(7) The medical operation system according to any one of (4) to (6), in which

the outer slave has a shape that allows stress to concentrate at the two positions at which the strain detection elements are disposed.

(8) The medical operation system according to (4), in which

the processing unit calculates a translational force and a moment acting on the end effector, by multiplying an average value of strain amounts detected by all the strain detection elements and a result of subtraction of the average value from detection values obtained from the respective strain detection elements, by a predetermined calibration matrix.

(9) The medical operation system according to (8), in which

the processing unit removes a strain component caused by a temperature change from the average value, and calculates a force acting in the long axis direction of the end effector.

(10) A surgical system including: a master device; and a slave device remotely controlled by the master device,

the slave device including

an inner slave having an end effector,

an outer slave into which the inner slave is inserted, the outer slave supporting the inner slave at a position that allows the end effector to protrude outward from an end of the outer slave,

a strain detection unit that detects strain generated in the outer slave,

a processing unit that calculates a force acting on the end effector in a living subject, on the basis of a result of detection performed by the strain detection unit, and

an output unit that outputs a result of processing performed by the processing unit, to the master device.

(11) A surgical instrument including:

an inner slave having an end effector;

an outer slave into which the inner slave is inserted, the outer slave supporting the inner slave at a position that allows the end effector to protrude outward from an end of the outer slave;

a strain detection unit that detects strain generated in the outer slave; and

a transmission unit that transmits a result of detection performed by the strain detection unit.

(12) An external force sensing system including:

an inner slave having an end effector;

an outer slave into which the inner slave is inserted, the outer slave supporting the inner slave at a position that allows the end effector to protrude outward from an end of the outer slave;

a strain detection unit that detects strain generated in the outer slave; and

a processing unit that calculates a force acting on the end effector, on the basis of a result of detection performed by the strain detection unit.

### REFERENCE SIGNS LIST

100 Surgical system

110 Gripping mechanism unit

111 End effector

112 Cable

120 Outer casing member

121 First outer casing

122 Second outer casing

123 First joint

124 Cable

125 Opening

126 Support

501a to 504a, 501b to 504b Strain detection element

801 to 804 Optical fiber

901, 902, 904 Dummy FBG sensor

1000 Signal processing unit

1001 Sum mode unit

1002 Difference mode unit

1003 Dummy FBG processing unit

1004 Translational force/moment derivation unit

1100 Robot system

1110 Master device

1111 Operation unit

1112 Conversion unit

1113 Communication unit

1114 Force sense presentation unit

1120 Slave device

1121 Drive unit

1122 Detection unit

1123 Communication unit

1. A medical operation system comprising:
an inner slave having an end effector;
an outer slave into which the inner slave is inserted, the outer slave supporting the inner slave at a position that allows the end effector to protrude outward from an end of the outer slave;
a strain detection unit that detects strain generated in the outer slave; and
a processing unit that calculates a force acting on the end effector in a living subject, on a basis of a result of detection performed by the strain detection unit.

2. The medical operation system according to claim 1, wherein
the outer slave has a bending portion that bends in a long axis direction, and
the strain detection unit is disposed on a distal end side than the bending portion.

3. The medical operation system according to claim 1, wherein
the outer slave has a structure decoupled from the inner slave, and a cable for pulling the end effector is inserted together with the inner slave into the outer slave.

4. The medical operation system according to claim 1, wherein
the strain detection unit includes strain detection elements disposed at two positions on respective opposite sides in two directions perpendicular to the long axis direction of the outer slave, and
the processing unit calculates a translational force and a moment acting on the end effector, on a basis of strains at the two positions on the respective opposite sides in the two directions perpendicular to the long axis direction of the outer slave, the strains having been detected by the strain detection elements.

5. The medical operation system according to claim 4, wherein
the strain detection unit includes the strain detection elements including FBG sensors formed at the two positions on optical fibers attached to the respective opposite sides in the two directions perpendicular to the long axis direction of the outer slave.

6. The medical operation system according to claim 5, wherein
dummy FBG sensors are formed in the optical fibers, and
the processing unit removes a strain component caused by a temperature change from a result of detection performed by the FBG sensors, on a basis of wavelength changes of the dummy FBG sensors.

7. The medical operation system according to claim 4, wherein
the outer slave has a shape that allows stress to concentrate at the two positions at which the strain detection elements are disposed.

8. The medical operation system according to claim 4, wherein
the processing unit calculates a translational force and a moment acting on the end effector, by multiplying an average value of strain amounts detected by all the strain detection elements and a result of subtraction of the average value from detection values obtained from the respective strain detection elements, by a predetermined calibration matrix.

9. The medical operation system according to claim 8, wherein
the processing unit removes a strain component caused by a temperature change from the average value, and calculates a force acting in the long axis direction of the end effector.

10. A surgical system comprising: a master device; and a slave device remotely controlled by the master device,
the slave device including
an inner slave having an end effector,
an outer slave into which the inner slave is inserted, the outer slave supporting the inner slave at a position that allows the end effector to protrude outward from an end of the outer slave,
a strain detection unit that detects strain generated in the outer slave,
a processing unit that calculates a force acting on the end effector in a living subject, on a basis of a result of detection performed by the strain detection unit, and
an output unit that outputs a result of processing performed by the processing unit, to the master device.

11. A surgical instrument comprising:
an inner slave having an end effector;
an outer slave into which the inner slave is inserted, the outer slave supporting the inner slave at a position that allows the end effector to protrude outward from an end of the outer slave;
a strain detection unit that detects strain generated in the outer slave; and
a transmission unit that transmits a result of detection performed by the strain detection unit.

12. An external force sensing system comprising:
an inner slave having an end effector;
an outer slave into which the inner slave is inserted, the outer slave supporting the inner slave at a position that allows the end effector to protrude outward from an end of the outer slave;
a strain detection unit that detects strain generated in the outer slave; and
a processing unit that calculates a force acting on the end effector, on a basis of a result of detection performed by the strain detection unit.
